(19) United States
(12) Patent Application Publication
(10) Pub. No.: US 2013/0261467 A1
(43) Pub. Date: Oct. 3, 2013

(54) METHOD FOR FORMING AN ULTRASOUND DEVICE, AND ASSOCIATED APPARATUS

(71) Applicant: RESEARCH TRIANGLE INSTITUTE, Research Triangle Park, NC (US)

(72) Inventors: David Dausch, Raleigh, NC (US); James Carlson, Durham, NC (US); Kristin Hedgepath Gilchrist, Durham, NC (US)

(21) Appl. No.: 13/906,700

(22) Filed: May 31, 2013

Related U.S. Application Data
(63) Continuation of application No. PCT/US2011/062625, filed on Nov. 30, 2011.
(60) Provisional application No. 61/419,534, filed on Dec. 3, 2010.

(51) Int. Cl. A61B 8/00; A61B 8/12

(52) U.S. Cl. CPC A61B 8/4494; A61B 8/12; A61B 8/445
USPC 600/466; 600/459; 29/25.35

(57) ABSTRACT

A method and apparatus directed to formation of a connection with an ultrasonic transducer apparatus (UTA) comprising a transducer device having first and second electrodes is provided. The UTA is engaged with an interposer device surface. The interposer device is greater in at least one lateral dimension than and extends laterally outward of the UTA, and comprises at least two laterally-extending conductors. A conductive engagement is formed between the first and second electrodes and respective first ends of the conductors. A connection support substrate is engaged with the interposer device about second ends of the conductors, and includes at least two connective elements for forming a conductive engagement with the respective second ends of the conductors. The UTA is then inserted into a catheter member lumen such that the device plane of the UTA and the at least two connective elements extend axially along the lumen.

### BACKGROUND OF THE DISCLOSURE

1. Field of the Disclosure

Aspects of the present disclosure relate to ultrasonic transducers, and, more particularly, to a method of forming a connection with a laterally-facing piezoelectric micromachined ultrasonic transducer housed in a catheter, and associated ultrasound apparatus.

2. Description of Related Art

Some micromachined ultrasonic transducers (MUTs) may be configured, for example, as a piezoelectric micromachined ultrasonic transducer (pMUT) as disclosed in U.S. Pat. No. 7,449,821 assigned to Research Triangle Institute, also the assignee of the present disclosure, which is also incorporated herein in its entirety by reference.

The formation of a pMUT device, such as the pMUT device defining an air-backed cavity as disclosed in U.S. Pat. No. 7,449,821, may involve the formation of an electrically-conductive connection between the first electrode (i.e., the bottom electrode) of the transducer device, wherein the first electrode is disposed on the front side of the substrate opposite to the air-backed cavity of the pMUT device, and the conformal metal layer(s) applied to the air-backed cavity for providing subsequent connectivity, for example, to an integrated circuit (“IC”) or a flex cable.

In some instances, one or more pMUTs, for example, arranged in a transducer array, may be incorporated into the end of an elongate catheter or endoscope. In those instances, for a forward-looking arrangement, the transducer array of pMUT devices must be arranged such that the plane of the piezoelectric element of each pMUT device is disposed perpendicularly to the axis of the catheter/endoscope. This configuration may thus limit the lateral space about the transducer array, between the transducer array and the catheter wall, through which signal connections may be established with the front side of the substrate. Further, directing such signal connections laterally to the transducer array to the front side thereof, may undesirably and adversely affect the diameter of the catheter (i.e., a larger diameter catheter may undesirably be required in order to accommodate the signal connections passing about the transducer array).

Where the transducer array is a one-dimensional (1D) array, external signal connections to the pMUT devices may be accomplished by way of a flex cable spanning the series of pMUT devices in the transducer array so as to be in electrical engagement with (i.e., bonded to) each pMUT device via the conformal metal layer thereof. For instance, As shown in FIG. 1A, in one exemplary 1D transducer array 100 (e.g., 1×64 elements), pMUT devices forming the array elements 120 may be attached directly to a flex cable 140, with the flex cable 140 including one electrically-conductive signal lead per pMUT device, plus a ground lead. For a forward-looking transducer array, the flex cable 140 is bent about the opposing ends of the transducer array such that the flex cable 140 can be routed through the lumen of the catheter/endoscope which, in one instance, may comprise an ultrasound probe. However, for a forward-looking transducer array in a relatively small catheter/endoscope, such an arrangement may be difficult to implement due to the severe bend requirement for the flex cable (i.e., about 90 degrees), which may also be compounded by the number of conductors comprising the flex cable and the engagement of the electrically-conductive signal leads to the pMUT devices (also about a bend of about 90 degrees), in order for the transducer array to be disposed within the lumen of the catheter/endoscope.

Further, for a forward-looking two-dimensional (2D) transducer array, signal interconnection with the individual pMUT devices may also be difficult. That is, for an exemplary 2D transducer array (e.g. 14×14 to 40×40 elements), there may be many more required signal interconnections with the pMUT devices, as compared to a 1D transducer array. As such, more wires and/or multilayer flex cable assemblies may be required to interconnect with all of the pMUT devices in the transducer array. However, as the number of wires and/or flex cable assemblies increases, the more difficult it becomes to bend the larger amount of signal interconnections about the ends of the transducer device to achieve the 90 degree bend required to integrate the transducer array into a catheter/endoscope. In addition, the pitch or distance between adjacent pMUT devices may be limited due to the required number of wires/conductors. Accordingly, such limitations may undesirably limit the minimum size (i.e., diameter) of the catheter/endoscope that can readily be achieved.

Co-pending U.S. Patent Application No. 61/329,258 (Methods for Forming a Connection with a Micromachined Ultrasonic Transducer, and Associated Apparatuses; filed Apr. 29, 2010, and assigned to Research Triangle Institute, also the assignee of the present application), discloses improved methods of forming an electrically-conductive connection between a pMUT device and, for example, an integrated circuit (“IC”), a flex cable, or a cable assembly, wherein individual signal leads extend parallel to the operational direction of the transducer array or perpendicularly to the transducer array face to engage the respective pMUT devices in the transducer array (see generally, e.g., FIG. 1B). Furthermore, the '258 application discloses that additional signal processing integrated circuits (IC's) can be integrated between the transducer array and the corresponding connective elements, thereby increasing the dimension of the transducer/connective element stack in a longitudinal direction of the disposition thereof in the catheter, but not increasing the lateral spacing around the transducer array, thus facilitating the configuration of the catheter to achieve a minimal diameter for a forward-looking transducer array configuration.

In the case of side- or lateral-looking transducer arrays, the transducer array is arranged such that the plane of the piezoelectric element of each transducer device is disposed in parallel to the axis of the catheter/endoscope. In such instances, there is relatively more lateral space about the transducer array, between the transducer array and the catheter wall, along the length of the transducer array, which may be used to attach connective elements thereto. However, the space between the back side of the transducer array and the catheter wall may be limited, particularly, for example, in catheters having an inner diameter of about 3 mm or less. Further, the previously-noted thicker stacks placed, for example, in a transducer element as illustrated in FIG. 1B, and including a transducer array, signal processing IC's and connective elements, may not necessarily be feasible in instances of the limited catheter inner diameter. Such a configuration may also undesirably impart mechanical stresses to the signal lead (which must be bent about 90 degrees to be routed from the transducer and along the catheter) and/or the transducer array interface due to the thickness of the transducer/IC stack and the limited space available across the catheter diameter.

One particular example of a prior art side-looking ultrasound catheter transducer is shown in FIG. 2, wherein a piezoelectric element 200 may be attached to a flex cable 210 using conductive epoxy 220. A top electrode 230 and matching layer 240 may then be deposited on the piezoelectric element 200, and the structure is then diced using a saw, wherein the cuts extend down to the flex cable 210 in order to form the elements of the transducer array 250. An acoustic backing 260 may then be applied to the back of the flex cable 210. However, such a configuration may be limited with respect to the number of transducer elements that can be practically implemented due, for instance to the resolution limit of the signal traces of the flex cable. For example, for a 3 mm catheter, only 16 traces with 100 μm pitch (plus ground strips on each side) may fit laterally within the lumen of the catheter. As such, an appropriate flex cable, such as a Siemens AcuNav flex cable with 64 elements, may undesirably have to be folded into 4 layers of 16 traces each (plus grounds) to connect all of the elements of a 64 element transducer array. Further, for 2D transducer arrays, high element counts (e.g., 196 to 1,600 elements) may require multilayer flex cabling for attachment and interconnection of all transducer elements, further increasing cost and complexity of the flex cabling. Multilayer flex cable could require up to 16 levels to connect all transducer elements due to limitations, for example, related to the pitch of conductor traces and interlevel vias in the flex cable (i.e., typically having a minimum of 100 μm pitch or more, depending on the number of levels). A multiple level flex cable may thus be undesirably expensive, difficult (or impossible) to manufacture, and may not be robust due to a relatively high probability of short circuits in light of the increased number of metal levels and vias. Other disadvantages of multilayer flex cabling may include higher conductor impedance, higher insertion loss, greater cross coupling between element traces, and higher shunt-to-ground capacitance which may reduce penetration depth compared to coaxial cabling (though typical coaxial cabling cannot be made with sufficiently fine pitch to be used in such catheter applications). Flex cabling may also be typically limited to segments of approximately 1 foot in length. Thus for a catheter that is 3 feet in total length, multiple flex cable segments must be serially connected in order to complete the electrical connection through the entire catheter, thereby undesirably increasing complexity and cost of assembly.

Thus, there exists a need in the ultrasonic transducer art, particularly with respect to a piezoelectric micromachined ultrasound transducer (“pMUT”), whether having an air-backed cavity or not, for improved methods of forming an electrically-conductive connection between the pMUT device and, for example, an integrated circuit (“IC”) and/or corresponding connective elements. In addition, it would be desirable to reduce the thickness of a chip stack containing the transducer array, IC devices and flex cabling, wiring and/or connective elements such that the chip stack may be accommodated within the relatively small diameter of a catheter or endoscope in a side looking configuration, for example, in cardiovascular devices, intravascular ultrasound devices, or laparoscopic surgery devices. Furthermore, it would be desirable to provide a method for forming electrical connections with a transducer array having a relatively higher transducer element count/density that is cost efficient (i.e., relatively low cost) and relatively manufacturable. Such solutions should desirably be effective for 2D transducer arrays, particularly 2D pMUT transducer arrays, but should also be applicable to 1D transducer arrays, in forward-looking and/or side looking arrangements, and should desirably allow greater scalability in the size of the probe/catheter/endoscope having such transducer arrays integrated therein.

### BRIEF SUMMARY OF THE DISCLOSURE

The above and other needs are met by aspects of the present disclosure, wherein one such aspect relates to a method of forming an ultrasound device having an ultrasonic transducer apparatus comprising a transducer device defining a device plane, and including a piezoelectric material disposed between a first electrode and a second electrode. Such a method comprises engaging the ultrasonic transducer apparatus with a surface of an interposer device such that the device plane of the ultrasonic transducer apparatus is substantially parallel to the interposer device, wherein the interposer device is greater in at least one lateral dimension than the ultrasonic transducer apparatus so as to extend laterally outward thereof along the device plane upon engagement therewith, and comprises at least two conductors extending laterally therealong, with each conductor having opposed first and second ends. An electrically-conductive engagement is formed between each of the first and second electrodes and the first ends of the respective conductors, wherein at least one of the first and second ends of each conductor extends outwardly of a periphery of the ultrasonic transducer apparatus in the at least one greater lateral dimension of the interposer device. A connection support substrate is engaged with the interposer device about the second ends of the conductors and outwardly of the periphery of the ultrasonic transducer apparatus, wherein the connection support substrate has at least two connective elements operably engaged therewith, so as to form an electrically-conductive engagement between each connective element and the respective second ends of the conductors. The ultrasonic transducer apparatus, engaged with the interposer device and the connection support substrate, is then inserted into a lumen defined by a wall of a catheter member and about an end thereof, such that the device plane of the ultrasonic transducer apparatus extends parallel to the wall and such that the at least two connective elements extend along the lumen away from the end of the catheter member.

Another aspect of the present disclosure provides an ultrasound device, comprising an ultrasonic transducer apparatus including a transducer device defining a device plane, and having a piezoelectric material disposed between a first electrode and a second electrode. An interposer device has a surface configured to engage the ultrasonic transducer apparatus such that the device plane of the ultrasonic transducer apparatus is substantially parallel to the interposer device. The interposer device is greater in at least one lateral dimension than the ultrasonic transducer apparatus so as to extend laterally outward thereof along the device plane, and comprises at least two conductors extending laterally therealong, wherein each conductor has opposed first and second ends. The ultrasonic transducer apparatus is engaged with the interposer device so as to form an electrically-conductive engagement between each of the first and second electrodes and the first ends of the respective conductors, with at least one of the first and second ends of each conductor extending outwardly of a periphery of the ultrasonic transducer apparatus in the at least one greater lateral dimension of the interposer device. A connection support substrate is engaged with the interposer device about the second ends of the conductors and outwardly of the periphery of the ultrasonic transducer apparatus. The connection support substrate has at least two connective elements operably engaged therewith, and is engaged with the interposer device so as to form an electrically-conductive engagement between each connective element and the respective second ends of the conductors. A catheter member has a wall defining a lumen, wherein the lumen is configured to receive the ultrasonic transducer apparatus, engaged with the interposer device and the connection support substrate, about an end thereof, such that the device plane of the ultrasonic transducer apparatus extends parallel to the wall and such that the at least two connective elements extend along the lumen away from the end of the catheter member.

Aspects of the present disclosure thus address the identified needs and provide other advantages as otherwise detailed herein.

### BRIEF DESCRIPTION OF THE SEVERAL VIEWS OF THE DRAWING(S)

Having thus described the disclosure in general terms, reference will now be made to the accompanying drawings, which are not necessarily drawn to scale, and wherein:

FIGS. 1A and 1B schematically illustrate a prior art arrangements for forming a connection with a forward-looking transducer apparatus disposed in a lumen;

FIG. 2 schematically illustrates a prior art arrangement for forming a connection with a side-looking transducer apparatus disposed in a lumen;

FIGS. 3 and 4 schematically illustrate an arrangement for forming a connection with a side-looking one-dimensional piezoelectric micromachined ultrasonic transducer array, according to one aspect of the disclosure;

FIGS. 5A-5C schematically illustrate an arrangement for forming a connection support substrate for connection with a side-looking transducer apparatus, according to another aspect of the disclosure;

FIGS. 6A and 6B schematically illustrate side and top views of an arrangement for foliating a connection with a side-looking one- or two-dimensional piezoelectric micromachined ultrasonic transducer array, according to another aspect of the disclosure;

FIGS. 7A and 7B schematically illustrate side and top views of an arrangement for foliating a connection with a side-looking one- or two-dimensional piezoelectric micromachined ultrasonic transducer array, according to yet another aspect of the disclosure; and

FIGS. 8A and 8B schematically illustrate side and top views of a side-looking ultrasound apparatus, according to a further aspect of the disclosure.

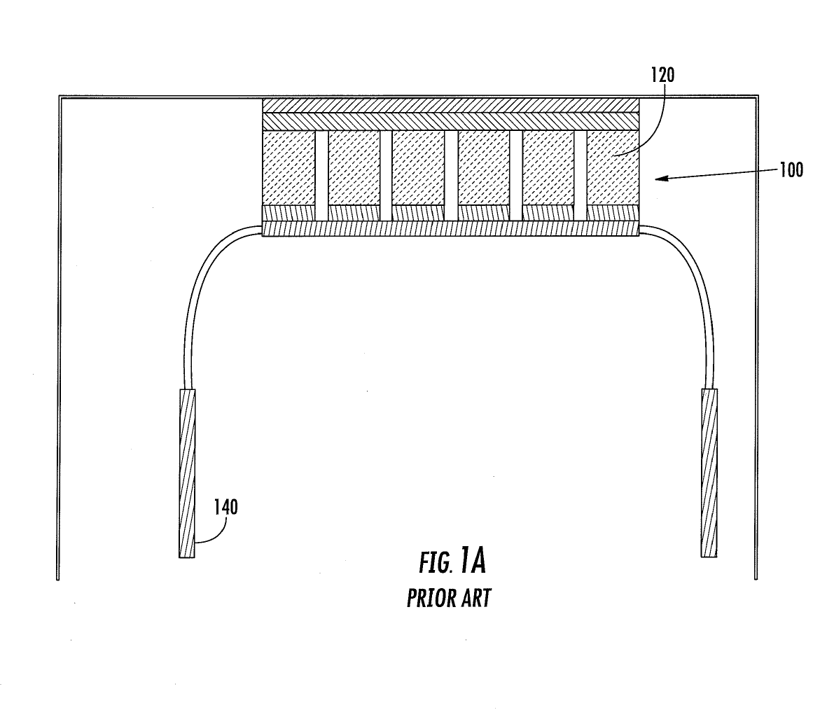

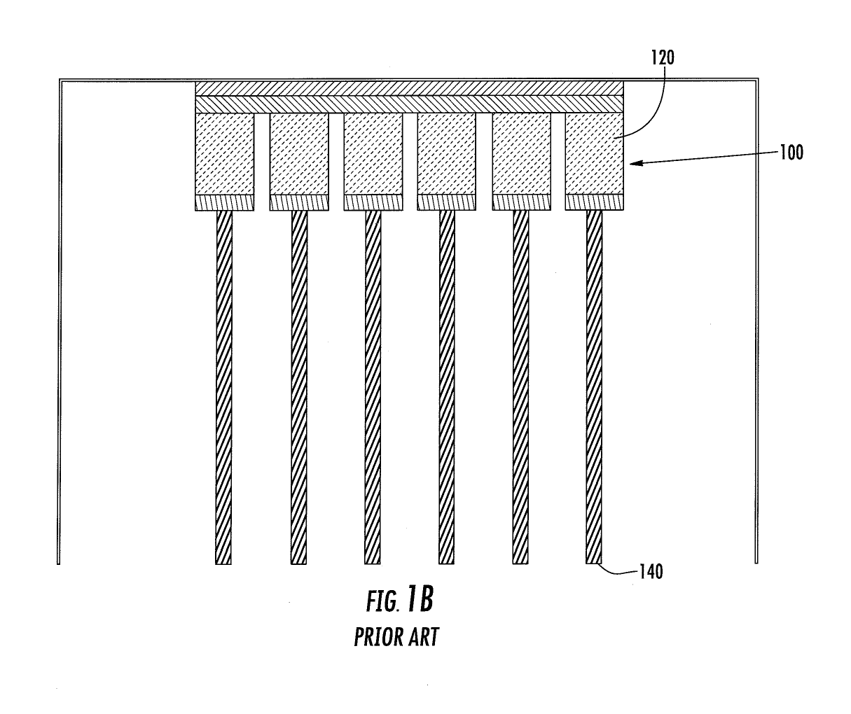

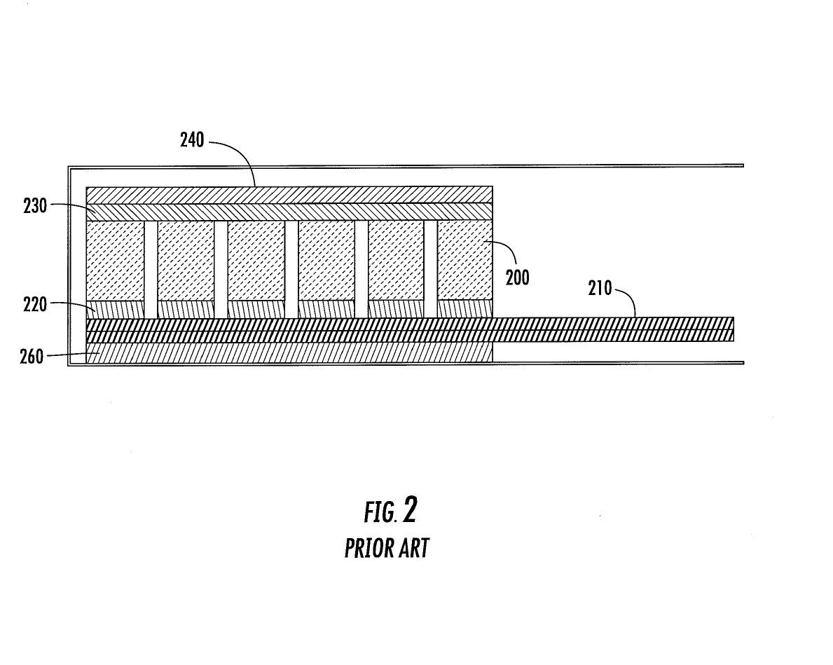

### DETAILED DESCRIPTION OF THE DISCLOSURE

The present disclosure now will be described more fully hereinafter with reference to the accompanying drawings, in which some, but not all aspects of the disclosure are shown. Indeed, the disclosure may be embodied in many different foams and should not be construed as being limited to the aspects set forth herein; rather, these aspects are provided so that this disclosure will satisfy applicable legal requirements. Like numbers refer to like elements throughout.

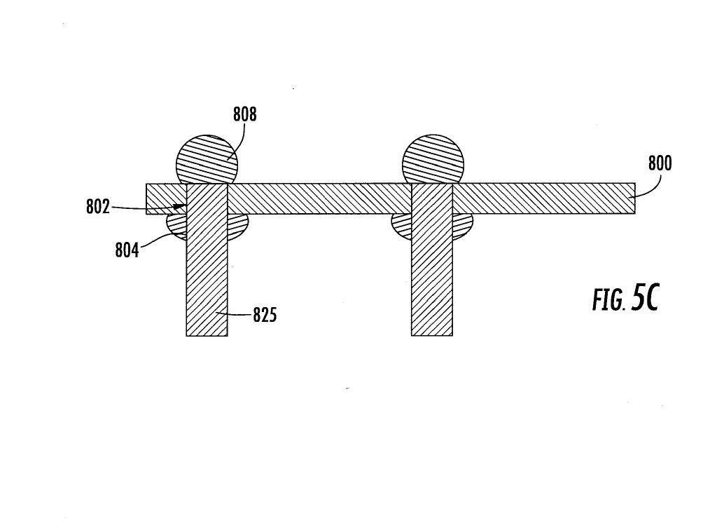

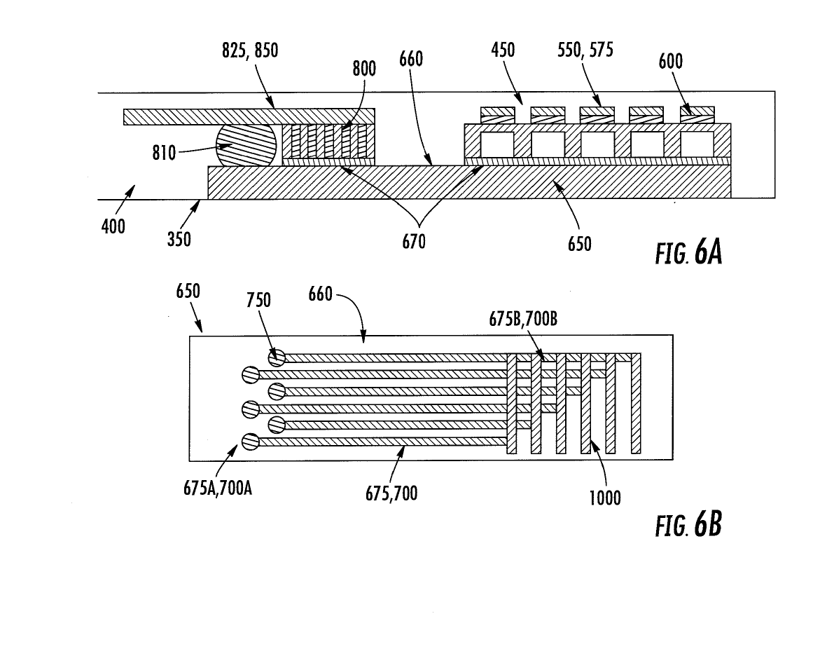

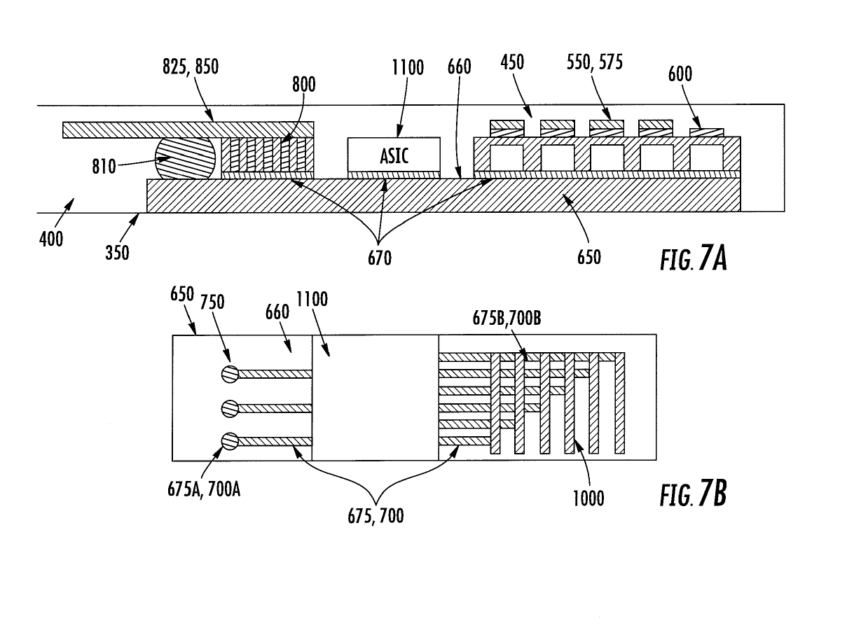

A representative ultrasound device 300, such as a catheter-based ultrasonic transducer array, is shown in FIG. 3. Such an exemplary aspect of the present disclosure includes a catheter member 350 defining an axially-extending lumen 400. In such an aspect, the lumen 400 houses an ultrasonic transducer apparatus 450, such as one or more transducer devices, which may be arranged in the form of a one-dimensional or two-dimensional transducer array. The ultrasonic transducer apparatus 450 defines a device plane 500, and each transducer device (see, e.g., FIGS. 6A and 7A) includes a piezoelectric material 550 disposed between a first electrode 575 and a second electrode 600. An interposer device 650 may also be disposed within the lumen 400. More particularly, the interposer device 650 includes a surface 660 configured to receive, engage and support the ultrasonic transducer apparatus 450 such that the device plane 500 of the ultrasonic transducer apparatus 450 is substantially parallel to the interposer device 650. The ultrasonic transducer apparatus 450 may be secured to the surface 660, for example, by a suitable adhesive or epoxy. In instances where the adhesive or other securement mechanism is involved in forming an electrically-conductive engagement between the ultrasonic transducer apparatus 450 and the surface 660, a conductive material such as, for example, an anisotropically-conductive epoxy may be used to secure the ultrasonic transducer apparatus 450 to the surface 660 of the interposer device 650. In some instances, the interposer device 650 may be comprised, for example, of silicon or other suitable material.

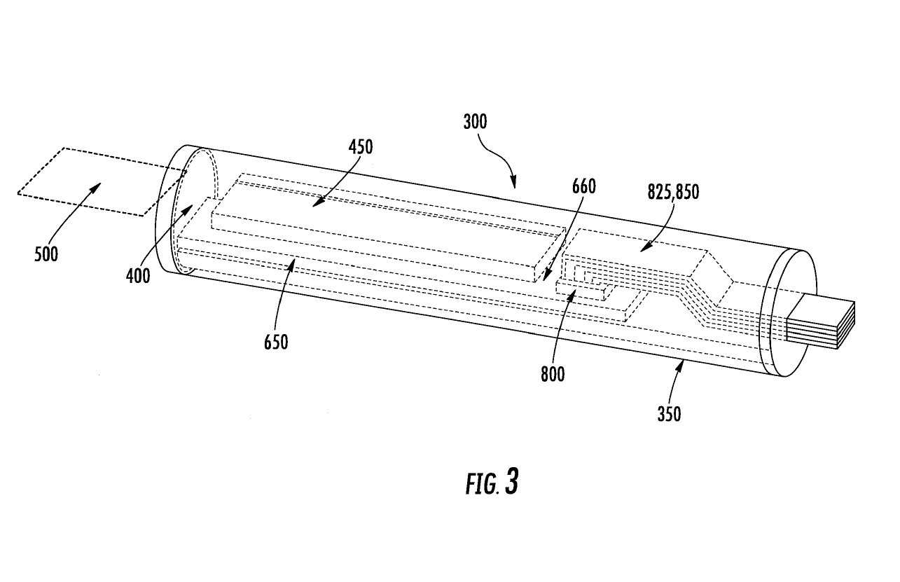

In one aspect, the interposer device 650 is greater in at least one lateral dimension than the ultrasonic transducer apparatus 450 (see, e.g., FIGS. 3 and 4) so as to extend laterally outward thereof along the device plane 500. In some instances, the interposer device 650 also includes at least two conductors 675, 700 (See, e.g., FIGS. 4, 6B, and 7B) extending laterally therealong, wherein the conductors 675, 700 have opposed first ends 675A, 700A and second ends 675B, 700B. The ultrasonic transducer apparatus 450 is engaged with the interposer device 650 so as to faun an electrically-conductive engagement between each of the first and second electrodes 575, 600 and the first ends 675A, 700A of the respective conductors 675, 700. In some aspects, either or both of the opposed ends of each conductor 675, 700 may extend in conjunction with the interposer device 650, outwardly of a periphery of the ultrasonic transducer apparatus 450 in the one or more greater lateral dimensions of the interposer device 650. That is, upon engagement of the ultrasonic transducer apparatus 450 with the interposer device 650, the interposer device 650 will extend outwardly of the periphery of the ultrasonic transducer apparatus 450 in at least one lateral direction. As such, either or both of the conductors 675, 700 may have one end thereof extending through the interposer device 650 to the interface between the interposer device 650 and the ultrasonic transducer apparatus 450, so as to form the electrically-conductive connection with the ultrasonic transducer apparatus 450, wherein such an aspect is disclosed in further detail herein.

In other aspects, either or both of the conductors 675, 700 may have one end thereof extending through the interposer device 650 so as to be exposed with respect to the surface of the interposer device 650 with which the ultrasonic transducer apparatus 450 is engaged, but outside the periphery of the ultrasonic transducer apparatus 450. In such instances, the electrodes 575, 600 may be electrically-engaged with the first end(s) 675A, 700A of the conductors 675, 700 by way of discrete conductive elements (not shown) engaged therebetween to respective wirebond pads 250A, 250B such as, for example in a wire bonding process. Further, in some aspects, the ultrasonic transducer apparatus 450 (i.e., pMUT), may or may not include metalized through-substrate interconnects connecting the first electrode 575 to the back side of the substrate. Accordingly, as shown in FIG. 4, in some aspects, the signal and ground traces of the transducer devices of the ultrasonic transducer apparatus 450 may be routed to the peripheral edges of the ultrasonic transducer apparatus 450 (i.e., into electrically-conductive engagement with wirebond pads 250A, 250B) and wirebonded to corresponding wirebond pads 250A, 250B in electrically-conductive engagement with the first and second conductors 675, 700 associated with the interposer device 650. Using such a configuration of the ultrasonic transducer apparatus 450, fewer photomask levels, for example, are used to fabricate the transducer devices, thus reducing fabrication costs. However, the footprint (lateral area) of the ultrasonic transducer apparatus 450 may be required to be larger to accommodate the wirebond pads. For instance, a 2 mm wide ultrasonic transducer device 450 (without metalized through-substrate interconnects) would require about a 2.8 mm to about a 3 mm wide interposer device 650, which would fit within the lumen of a 12 French (4 mm O.D.) catheter. However, using metalized through-substrate interconnects, instead of a wirebond pad configuration, such that an electrically-conductive engagement is formed with the conductors 675, 700 associated with the interposer device 650 by way of the conductive layer associated with the air-backed cavities of the transducer devices, the width of the ultrasonic transducer device 450 could be reduced to between about 1.7 mm and about 1.8 mm, and the interposer device 650 could also have substantially the same width, since the additional width required for wirebond pads is eliminated. In such an instance, the implementation of transducer devices with metalized through-substrate interconnects would reduce the required catheter size to 8 French (2.7 mm O.D.).

As disclosed, the ultrasonic transducer apparatus 450 may be secured to the surface 660, for example, by a bonding material 670 such as a suitable adhesive or epoxy. In instances where the adhesive or other securement mechanism is involved in forming an electrically-conductive engagement between the ultrasonic transducer apparatus 450 and the surface 660, a conductive material such as, for example, an anisotropically-conductive epoxy may be used to secure the ultrasonic transducer apparatus 450 to the surface 660 of the interposer device 650. In some instances, it may be desirable to implement an acoustically-absorbent epoxy such as, for example, a tungsten-filled epoxy, to secure the ultrasonic transducer apparatus 450 to the interposer device 650, which may also provide an acoustic backing for the transducer devices. If the ultrasonic transducer device 450 is wirebonded to the conductors 675, 700 associated with the interposer device 650, a potting epoxy may be used to cover the wirebond connections.

In some aspects, the conductors 675, 700 extend laterally with respect to the interposer device 650 such that the second ends 675B, 700B thereof are in electrically-conductive engagement with an array of electrically-conductive pads 750 (see, e.g., FIG. 4), wherein the interposer device 650 is configured to receive and engage a connection support substrate 800 such that the second ends 675B, 700B of the conductors 675, 700, via the pads 750, engage (in an electrically-conductive engagement) corresponding connective elements 825, 850 (see, e.g., FIG. 3) engaged with and supported by the connection support substrate 800 outwardly of the periphery of the ultrasonic transducer apparatus 450. The connective elements 825, 850 may comprise, for example, external signal leads for the ultrasonic transducer apparatus 450. As such, in some aspects, the ultrasonic transducer apparatus 450, engaged with the interposer device 650 and the connection support substrate 800, is configured to be received in an end portion of the lumen 400 defined by a wall of the catheter member 350, such that the device plane 500 of the ultrasonic transducer apparatus 450 extends parallel to the wall or axis of the catheter member 350 and such that the at least two connective elements 825, 850 extend along the lumen 400 away from the end of the catheter member 350 (i.e., so as to form a “side-looking” ultrasound device).

In some instances, the conductors 675, 700 associated with the interposer device 650 may be of different lengths due to the location and configuration of the corresponding wirebond pad with respect to the pads 750 for connecting with the connective elements 825, 850. As such, in some instances, the conductors 675, 700 associated with the interposer device 650 may be configured to have varying widths, or otherwise varying cross-sectional dimensions, such that differences between the electrical resistances of the conductors 675, 700 are minimized or substantially eliminated. That is, the conductors 675, 700 may be configured so as to achieve and maintain substantially constant impedance with respect to the signal leads extending to each transducer device of the ultrasonic transducer apparatus 450.

In some aspects, the connection support substrate 800 may be configured, for instance, to be compatible with a flip-chip aligner-bonder for facilitating engagement with the interposer device 650 supporting the ultrasonic transducer apparatus 450. As such, the interposer device 650 may advantageously be configured such that the arrangement of connective elements 825, 850 with respect to the connection support substrate 800 is not required to correspond to the arrangement of transducer devices in the array implemented by the ultrasonic transducer apparatus 450. For example, the pitch and/or gauge of the connective elements 825, 850 may be different from the pitch or electrode area of the transducer devices, wherein correspondence may be achieved, if necessary or desired, by appropriately configuring the conductors 675, 700 associated with the interposer device 650, as will be appreciated by one skilled in the art. Such a configuration of the interposer device 650 may be advantageous, for example, with respect to side-looking 1D (one-dimensional) arrays or ultrasonic transducer apparatuses 450. For instance, as shown in FIG. 4, a 5×16 array of wires/connective elements may be engaged with a 1×64 array of transducer devices in an ultrasonic transducer apparatus 450 through appropriate arrangement of the conductors associated with the interposer device 650. Accordingly, the implementation of such an interposer device may provide additional flexibility in the selection of cabling used (i.e., in the number of wires or connective elements per cable, as well as the wire pitch) for connection with the ultrasonic transducer apparatus 450, and may also allow the attachment of a wire/connective element array with larger number of wires (e.g., 8×16 or 128 wires) to provide additional ground leads to be interspersed between signal elements/wires to reduce noise and cross-talk between conductive elements.

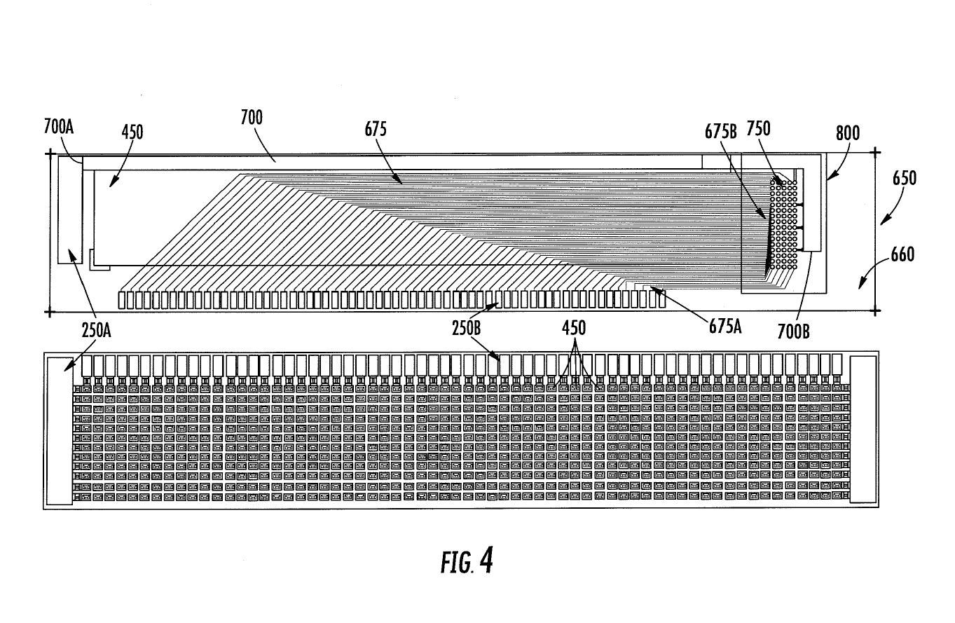

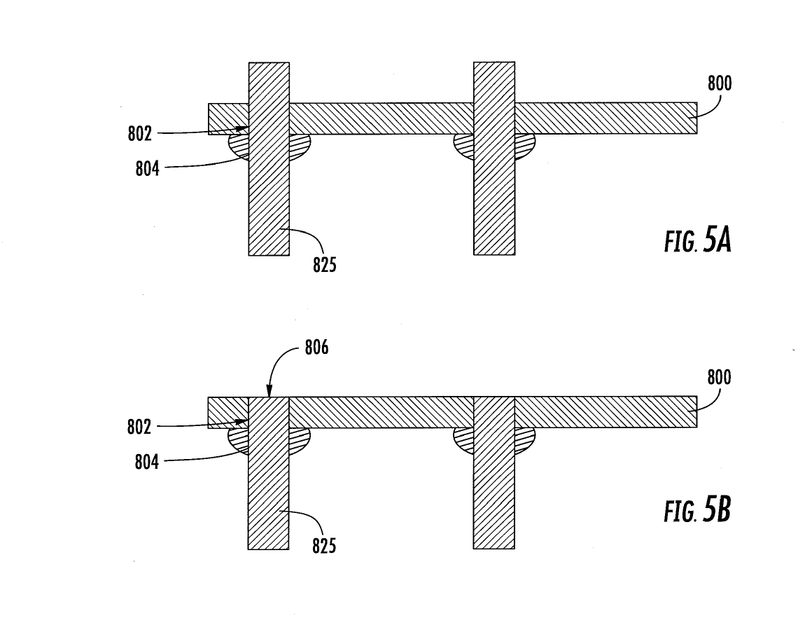

FIG. 5A schematically illustrates another aspect of the present disclosure directed to the formation of the connection support substrate 800 and subsequent connection thereof to the interposer device 650. More particularly, the connection support substrate 800 (comprised, for example, of silicon) is first etched, for example, using a DRIE process, to define a via 802 extending therethrough with sidewalls substantially perpendicular to the etched surface. The connection support substrate 800 may then be thermally oxidized to provide electrical isolation between adjacent vias (not shown). One of the connective elements (e.g., element 825) is then inserted into the via 802 so as to extend therethrough, and the connective element 825 then bonded to the connection support substrate 800 with a bonding material 804, such as a non-conductive epoxy, applied around the connective element 825 on the surface of the connection support substrate 800 opposite the surface of the connection support substrate 800 through which the connective element 825 extends. For example, fine gauge (e.g., 40-50 AWG) wire may be fed into the via and then potted within the via with a low-viscosity epoxy in a vacuum chamber to fill the voids. In some instances, the connective element 825 may comprise an elongate conductor circumscribed by an insulator. In such instances, the insulator may be configured to provide electrical isolation between the conductor/connective element 825 and the connection support substrate 800. In other instances, if the connective element 825 does not include the insulator, an insulator material (not shown) may be first deposited on the connection support substrate 800 so as to extend through the via 802, so as to electrically isolate the connective element 825 from the connection support substrate 800.

As shown in FIG. 5B, once the connective element 825 is secured to the connection support substrate 800, the surface of the connection support substrate 800 through which the connective element 825 extends is planarized, for example, by a mechanical polishing process or a chemical-mechanical polishing (CMP) process to produce a substantially planar surface having the end 806 of the connective element 825 exposed. In some instances, any gap between the connective element 825 and the wall defining the via 802 can be filled, for example, with a non-conductive epoxy to provide a void-free, planar surface of the connection support substrate 800 for subsequent processing. For instance, one aspect implements a microribbon cable, which includes individually insulated 46-48 AWG Cu wires with a Cu backplane under each ribbon to reduce cross talk. The microribbon cable can be fed one row at a time into the connection support substrate 800 rather than individual wires being guided into individual vias. The connective element 825 and/or the connection support substrate 800 is subsequently bonded to the interposer device 650 and/or the pads 750 associated therewith. In one such aspect, the conductive bonding material 808 may comprise, for example, a solder bump, as shown in FIG. 5C. In such instances, the bonding may be effectuated by reflowing the solder comprising the solder bump. In another aspect, the conductive bonding material 808 may comprise a metal (i.e., Au, Al, or Cu) or plated metal stud bumps formed using a wire bonder or by electroplating, wherein such stud bumps can be thermo-compression bonded to provide the electrically-conductive engagement through direct metal bonding. An anisotropic conductive epoxy may also be implemented as the conductive bonding material 808. Alignment of the connective elements 825, 850 associated with the connection support substrate 800 with the pads 750 associated with the interposer device 650 can be accomplished, for example, using a flip-chip aligner-bonder. Once bonded to the pads 750, the connective elements 825, 850 are bent about 90 degrees so as to extend substantially parallel to the device plane 500 (but such that the interface between the pads 750 and the connective elements 825, 850 extends perpendicularly to the device plane 500) so as to extend along the lumen 400 of the catheter member 350, as shown, for example, in FIGS. 6A and 7A. In some aspects, a strain relief element 810, such as additional epoxy, as shown, for instance, in FIGS. 6A and 7A may be applied between the connective elements 825, 850 and the interposer device 650 for relieving strain on the interface between the connection support substrate 800 and the interposer device 650 (as well as the interface between the pads 750 and the connective elements 825, 850).

Other aspects of the present disclosure are provided in FIGS. 6A and 6B, wherein the ultrasonic transducer apparatus 450 may comprise, for example, a vertically-integrated 1D or 2D transducer array (i.e., pMUT transducer devices with through-substrate interconnects). In such instances, both the first and second electrodes 575, 600 may be accessible with respect to one surface of the ultrasonic transducer apparatus 450. Accordingly, the ultrasonic transducer apparatus 450 may be directly engaged (i.e., without wirebonding) with the interposer device 650, without requiring the additional area or larger lateral dimension (with respect to both the ultrasonic transducer apparatus 450 and the interposer device 650) for wirebond pads and associated routing of conductors associated therewith. In such instances, the interposer device 650 may further comprise at least one electrically-conductive trace 1000 engaged with the surface 660 of the interposer device 650, wherein the trace(s) 1000 are configured to be in electrically-conductive engagement with the first ends 675A, 700A of the respective conductors 675, 700.

In some aspects, the ultrasonic transducer apparatus 450 may be engaged with the interposer device 650 such that an electrically-conductive engagement is formed between one of the first and second electrodes 575, 600 and the corresponding trace(s) 1000 using a bonding material 670 such as, for example, a conductive solder element, a conductive stud element, and a conductive bonding material disposed therebetween. For instance, the ultrasonic transducer apparatus 450 can be engaged with the surface 660 of the interposer device 650 using an anisotropic conductive epoxy, solder bumps, gold stud bumps or direct-plated metal bonding. The connection support substrate 800 may be engaged with the interposer device 650 in a similar manner via a bonding material 670 so as to form the electrically-conductive engagement between the conductors 675, 700 and the connective elements 825, 850.

Since, in some aspects, the interposer device 650 may be comprised of silicon, the conductors 675, 700 and/or the trace(s) 1000 may be formed using various semiconductor processing techniques, as will be appreciated by one skilled in the art. For example, conductive material may be deposited on the interposer device 650 and patterned by photolithography and etching, or lift-off processing. Once the conductive material is deposited and the conductors 675, 700 and/or the trace(s) 1000 formed, an insulator such as SiO₂ may be selectively deposited over the conductors 675, 700 and/or the trace(s) 1000 so as to prevent lateral electrical conduction, for instance, when an anisotropic conductive epoxy is used to engage the ultrasonic transducer apparatus 450 with the interposer device 650. In other instances, the deposition of the insulator over the conductors 675, 700 and/or the trace(s) 1000 may also prevent electrical conduction between the portions of the conductors 675, 700 and/or the trace(s) 1000 extending along the interposer device 650 under the interface between the ultrasonic transducer apparatus 450 and the interposer device 650.

The pads 750, conductors 675, 700, and trace(s) 1000 may be formed as different metallization levels with respect to the interposer device 650, with an insulator deposited between levels for electrical isolation. For example, the conductors 675, 700 connecting the pads 750 to the trace(s) 1000 may be formed as a first metallization level within the interposer device 650, while the pads 750 and/or the trace(s) 1000 may be formed as a second metallization level that may remain exposed about the surface 660. The exposed portions of the trace(s) 1000 may be implemented for direct connection to one of the electrodes of the ultrasonic transducer apparatus 450 or, in the case of a pMUT having an air-backed cavity, the electrodes 575, 600 on one side of the ultrasonic transducer apparatus 450. In some instances, connection of the second electrode 600 to the trace(s) 1000 could be accomplished by way of a conformal metallization layer deposited in the via comprising the air-backed cavity of the pMUT (not shown). In other instances, the smaller exposed pads (not shown) could be provided at the second ends 675B, 700B of the conductors 675, 700, wherein a transducer device of the ultrasonic transducer apparatus 450 could be electrically-engaged with the conductors 675, 700 via the small pads. In some instances, the small exposed pads could comprise a portion of the respective conductors, and may eliminate multiple level metallization requirements. However, in some aspects, as the required number of signal leads increases, it may be advantageous to include multiple levels of metallization within the interposer device 650. For example, for a 2D transducer array, 3-4 metallization levels associated with the interposer device 650 may be required for a transducer element count of between about 200 and about 400 elements, which may be advantageous, for instance, over a flex cable approach for connection to a 2D transducer array comprising the ultrasonic transducer device 450, which may require up to 16 flex cable levels due to the limitations of the available conductor pitch, typically on the order of 100 μm. In this regard, a 16-level multilayer flex cable may be too expensive, difficult to manufacture, and may not be sufficiently robust due to high probability of shorts. Smaller conductor pitch of between about 10 μm and about 50 μm could be fabricated, for example, on a silicon interposer device using silicon photolithography techniques having improved resolution.

In some aspects, as shown in FIGS. 7A and 7B, the ultrasonic transducer device 450 (i.e., a pMUT transducer device) disclosed herein, as necessary or desired, may be engaged with an IC or integrated circuit (e.g., a control IC such as an amplifier, multiplexer, or beam former) 1100, for example, via the interposer device 650. For instance, the IC 1100 could be engaged with the interposer device 650/conductors 675, 700, between the ultrasonic transducer device 450 and the connection support substrate 800 using, for example, solder bumps, gold stud bumps, metal stud bumps, anisotropic conductive epoxy, or other suitable electrically-conductive connection provisions. In one example, the IC 1100 may be configured as an application specific integrated circuit (ASIC), and the interposer device 650 may thus be configured to facilitate the integration of the ASIC within close proximity to the ultrasonic transducer apparatus 450. ASIC functions that could be integrated with respect to the IC 1100 in engagement with the interposer device 650 include, for example, amplification to enhance the small receive voltages generated by the transducer (pMUT) elements/devices within the array, multiplexing or switching for toggling transducer elements/devices between transmit mode and receive mode, timing or beam forming for facilitating receipt of the receive signals by the ultrasound system, and/or multiplexing of transmit and receive channels to reduce the number of required conductors from one element per conductor to multiple elements per conductor. In other instances, the IC 1100 may be configured as charge pump transmit circuits for generating relatively higher transmit voltages from a relatively small control signal sent from the ultrasound system (for example, the IC 1100 may comprise a multiplexer, an amplifier, a beam former, and/or a high voltage transmit circuit). Such ASIC functions may improve the performance of the ultrasonic transducer apparatus 450 (e.g., amplify receive signals prior to transmission on high-capacitance system cabling) and/or reduce the number of connective elements required to be housed within the catheter (e.g., 4:1 or 8:1 multiplexing of element transmit and/or receive signals by an appropriately-configured IC 1100). In such aspects, the interposer device 650 and the conductors 675, 700 therein, may be configured similarly to the arrangement for receiving the ultrasonic transducer apparatus 450 (i.e., with exposed conductive pads in communication with the conductors 675, 700), in order to facilitate integration of the IC 1100 (or multiple IC's) in communication with the ultrasonic transducer apparatus 450 and the pads 750/connective elements 825, 850 via the connection support substrate 800.

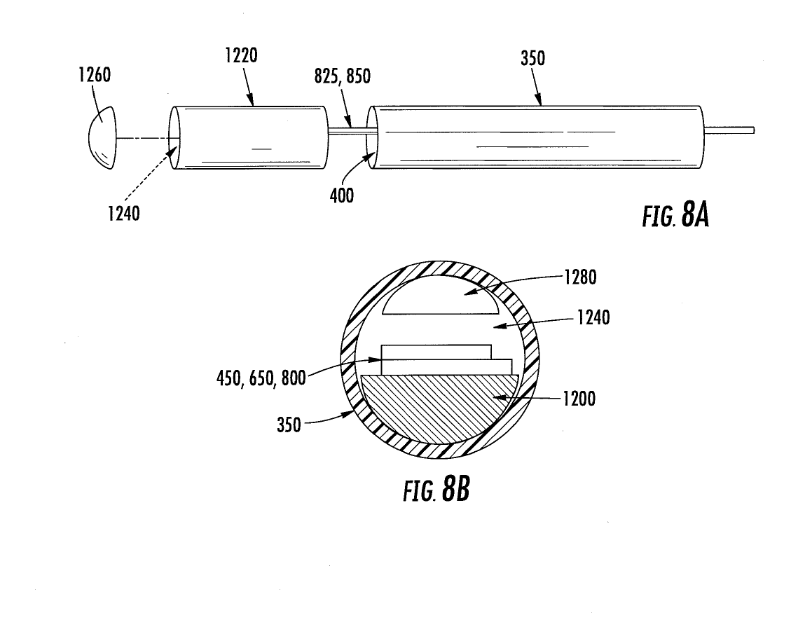

Many modifications and other aspects of the disclosures set forth herein will come to mind to one skilled in the art to which these disclosures pertain having the benefit of the teachings presented in the foregoing descriptions and the associated drawings. For example, the exemplary methods and aspects thereof as disclosed herein may also have related apparatuses associated therewith, as otherwise disclosed herein. As such, the apparatuses and methods disclosed herein may be suitably adapted to address such instances, within the scope of the present disclosure. Further, in another aspect regarding transducer (pMUT) arrays in a side-looking catheter, as shown in FIGS. 8A and 8B, the ultrasonic transducer apparatus 450, interposer device 650, and connection support substrate 800 may be mounted on a catheter mount 1200 inside a catheter transducer tip 1220, which may be configured (sized) to house the interposer device 650 and the connection support substrate 800 lengths (i.e., ˜2 cm). For example, the interposer device 650 may be about 14.5 mm in length for a 64 element 1D transducer (pMUT) array, wherein the array length may be about 10.5 mm. For a 2D transducer (pMUT) array with ˜200 elements and 2 mm×2 mm size, the interposer device 650 could be about 6 mm in length. The catheter transducer tip 1220 may be sealed at the opposing distal and proximal ends thereof, while being filled with an acoustic coupling fluid 1240 such as, for example, glycerin, polyethylene glycol or silicone oil. The conductive elements (i.e., microribbon or other cabling) extends through the proximal end of the catheter transducer tip 1220 and along the lumen 400 defined by the catheter member 350, and may terminate at an electronic device, such as a circuit board (not shown), about the proximal end of the catheter member 350. About the distal end of the catheter member 350, a rounded catheter cap 1260 may be engaged with or formed in the catheter transducer tip 1220 in order to facilitate insertion of the catheter member 350 during the medical procedure, such as an intracardiac or intravascular imaging process. The catheter transducer tip 1220 may also include an acoustic lens 1280 engaged with the wall of the catheter member 350 defining the lumen 400, opposite to the ultrasonic transducer apparatus 450. A passive lens may be implemented to improve image resolution for 1D transducer arrays (i.e., 1 element only in elevation), since such 1D arrays may not be capable of elevation focusing, whereas a 2D transducer array may have elevation focusing capabilities, which may thus not require a lens. The catheter member 350 may be comprised, for example, of Pebax™ or any other suitable materials exhibiting, for instance, low acoustic impedance and low absorption, which may be particularly beneficial for the wall of the catheter transducer tip 1220, which requires acoustic transmission capabilities for the ultrasonic transducer apparatus 450. The remaining portion of the catheter member 350 may also be comprised of Pebax™ or other suitable material exhibiting an appropriate elastic modulus and/or Shore hardness, for example, to provide flexibility near the distal catheter tip for steerability of the tip and rigidity in the catheter shaft proximal to the tip for pushability of the catheter member 350 through the body of the patient. Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

That which is claimed:

1. A method of forming an ultrasound device having an ultrasonic transducer apparatus comprising a transducer device defining a device plane, and including a piezoelectric material disposed between a first electrode and a second electrode, said method comprising:
engaging the ultrasonic transducer apparatus with a surface of an interposer device such that the device plane of the ultrasonic transducer apparatus is substantially parallel to the interposer device, the interposer device being greater in at least one lateral dimension than the ultrasonic transducer apparatus so as to extend laterally outward thereof along the device plane upon engagement therewith, and comprising at least two conductors extending laterally therealong, each conductor having opposed first and second ends;
forming an electrically-conductive engagement between each of the first and second electrodes and the first ends of the respective conductors, at least one of the first and second ends of each conductor extending outwardly of a periphery of the ultrasonic transducer apparatus in the at least one greater lateral dimension of the interposer device; and
engaging a connection support substrate with the interposer device about the second ends of the conductors and outwardly of the periphery of the ultrasonic transducer apparatus, the connection support substrate having at least two connective elements operably engaged therewith, so as to form an electrically-conductive engagement between each connective element and the respective second ends of the conductors.

2. A method according to claim 1, further comprising inserting the ultrasonic transducer apparatus, engaged with the interposer device and the connection support substrate, into a lumen defined by a wall of a catheter member and about an end thereof, such that the device plane of the ultrasonic transducer apparatus extends parallel to the wall and such that the at least two connective elements extend along the lumen away from the end of the catheter member.

3. A method according to claim 1, further comprising engaging the transducer device with a device substrate to form the ultrasonic transducer apparatus, the transducer device being configured with the first and second electrodes extending laterally with respect to the device substrate and to respective bond pads disposed about a periphery thereof.

4. A method according to claim 3, wherein engaging the ultrasonic transducer apparatus with a surface of an interposer device further comprises engaging the ultrasonic transducer apparatus with a surface of an interposer device with a non-conductive adhesive material disposed therebetween.

5. A method according to claim 3, wherein foaming an electrically-conductive engagement further comprises engaging a conductive member between the bond pads associated with each of the first and second electrodes and the first ends of the respective conductors.

6. A method according to claim 1, wherein the transducer device is disposed on a substrate and in communication with through-substrate interconnects, and wherein forming an electrically-conductive engagement further comprises forming an electrically-conductive engagement between each of the first and second electrodes and the first ends of the respective conductors via the through-substrate interconnects.

7. A method according to claim 6, wherein forming an electrically-conductive engagement between each of the first and second electrodes and the first ends of the respective conductors via one of a conductive solder element, a conductive stud element, and a conductive bonding material between the through-substrate interconnects and the first ends of the respective conductors.

8. A method according to claim 1, further comprising engaging the at least two connective elements with a connection support substrate, with the at least two connective elements inserted into and extending through respective vias defined by the connection support substrate, prior to engaging the connection support substrate with the interposer device.

9. A method according to claim 8, further comprising depositing an insulator material on the connection support substrate such that the insulator material extends along the vias defined thereby, prior to engaging the at least two connective elements with the connection support substrate.

10. A method according to claim 1, wherein engaging a connection support substrate with the interposer device comprises engaging a connection support substrate with the interposer device with one of a conductive solder element, a conductive stud element, and a conductive bonding material therebetween, so as to form an electrically-conductive engagement between each connective element and the respective second ends of the conductors.

11. A method according to claim 1, wherein engaging the connection support substrate with the interposer device further comprises engaging the connection support substrate with the interposer device such that the electrically-conductive engagement between each connective element and the respective second ends of the conductors extends substantially perpendicularly to the device plane of the ultrasonic transducer apparatus.

12. A method according to claim 1, further comprising engaging a strain relief device between the at least two connective elements and the interposer device so as to relieve strain on the electrically-conductive engagement between each connective element and the respective second ends of the conductors.

13. A method according to claim 1, wherein the interposer device further comprises at least one electrically-conductive trace engaged with the surface of the interposer device and in electrically-conductive engagement with the first ends of the respective conductors, and wherein forming an electrically-conductive engagement further comprises forming an electrically-conductive engagement between one of the first and second electrodes and the at least one trace with a conductive bonding material therebetween upon engaging the ultrasonic transducer apparatus with the surface of the interposer device.

14. A method according to claim 13, further comprising engaging at least one integrated circuit device with the interposer device between the ultrasonic transducer apparatus and connection support substrate, such that the at least one integrated circuit device is in electrically-conductive communication with at least one of the conductors.

15. A method according to claim 14, wherein engaging an integrated circuit device with the interposer device further comprises forming an electrically-conductive engagement between the integrated circuit device and the at least one trace with one of a conductive solder element, a conductive stud element, and a conductive bonding material therebetween.

16. A method according to claim 1, further comprising varying a cross-sectional dimension of each of the at least two conductors extending through the interposer device such that a resistance of each of the at least two conductors is substantially the same.

17. An ultrasound device, comprising:
an ultrasonic transducer apparatus comprising a transducer device defining a device plane, and including a piezoelectric material disposed between a first electrode and a second electrode:
an interposer device having a surface configured to engage the ultrasonic transducer apparatus such that the device plane of the ultrasonic transducer apparatus is substantially parallel to the interposer device, the interposer device being greater in at least one lateral dimension than the ultrasonic transducer apparatus so as to extend laterally outward thereof along the device plane, and comprising at least two conductors extending laterally therealong, each conductor having opposed first and second ends, the ultrasonic transducer apparatus being engaged with the interposer device so as to form an electrically-conductive engagement between each of the first and second electrodes and the first ends of the respective conductors, with at least one of the first and second ends of each conductor extending outwardly of a periphery of the ultrasonic transducer apparatus in the at least one greater lateral dimension of the interposer device;
a connection support substrate engaged with the interposer device about the second ends of the conductors and outwardly of the periphery of the ultrasonic transducer apparatus, the connection support substrate having at least two connective elements operably engaged therewith, the connection support substrate being engaged with the interposer device so as to form an electrically-conductive engagement between each connective element and the respective second ends of the conductors.

18. A device according to claim 17, further comprising a catheter member having a wall defining a lumen, the lumen being configured to receive the ultrasonic transducer apparatus, engaged with the interposer device and the connection support substrate, about an end thereof such that the device plane of the ultrasonic transducer apparatus extends parallel to the wall and such that the at least two connective elements extend along the lumen away from the end of the catheter member.

19. A device according to claim 17, wherein the ultrasonic transducer apparatus further comprises a device substrate engaged with the transducer device, and wherein the transducer device is configured with the first and second electrodes extending laterally with respect to the device substrate and to respective bond pads disposed about a periphery thereof.

20. A device according to claim 19, wherein the ultrasonic transducer apparatus is engaged with the surface of the interposer device with a non-conductive adhesive material disposed therebetween.

21. A device according to claim 19, further comprising a conductive member engaged between the bond pads associated with each of the first and second electrodes and the first ends of the respective conductors, and forming the electrically-conductive engagement therebetween.

22. A device according to claim 17, wherein the transducer device is disposed on a substrate and in communication with through-substrate interconnects, and wherein the through-substrate interconnects are configured to form an electrically-conductive engagement between each of the first and second electrodes and the first ends of the respective conductors.

23. A device according to claim 22, wherein the through-substrate interconnects are engaged with the first ends of the respective conductors with one of a conductive solder element, a conductive stud element, and a conductive bonding material therebetween.

24. A device according to claim 17, wherein the at least two connective elements are configured to be inserted into and to extend through respective vias defined by the connection support substrate.

25. A device according to claim 24, further comprising an insulator material deposited on the connection support substrate such that the insulator material extends along the vias defined thereby.

26. A device according to claim 17, wherein the connection support substrate is engaged with the interposer device with one of a conductive solder element, a conductive stud element, and a conductive bonding material therebetween, the one of the conductive solder element, the conductive stud element, and the conductive bonding material foaming an electrically-conductive engagement between each connective element and the respective second ends of the conductors.

27. A device according to claim 17, wherein the connection support substrate is engaged with the interposer device such that the electrically-conductive engagement between each connective element and the respective second ends of the conductors extends substantially perpendicularly to the device plane of the ultrasonic transducer apparatus.

28. A device according to claim 17, further comprising a strain relief device engaged between the at least two connective elements and the interposer device so as to relieve strain on the electrically-conductive engagement between each connective element and the respective second ends of the conductors.

29. A device according to claim 17, wherein the interposer device further comprises at least one electrically-conductive trace engaged with the surface of the interposer device and in electrically-conductive engagement with the first ends of the respective conductors, and wherein one of the first and second electrodes is arranged in electrically-conductive engagement with the at least one trace with a conductive bonding material disposed therebetween.

30. A device according to claim 29, further comprising at least one integrated circuit device engaged with the interposer device between the ultrasonic transducer apparatus and connection support substrate, such that the at least one integrated circuit device is in electrically-conductive communication with at least one of the conductors.

31. A device according to claim 30, wherein the at least one integrated circuit comprises one of a multiplexer, an amplifier, a beamformer, and a high voltage transmit circuit.

32. A device according to claim 30, wherein the integrated circuit device is arranged in electrically-conductive engagement with the at least one trace with one of a conductive solder element, a conductive stud element, and a conductive bonding material therebetween.

33. A device according to claim 17, wherein each of the at least two conductors extending through the interposer device is configured to include a varied cross-sectional dimension such that a resistance of each of the at least two conductors is substantially the same.
